(19) United States
(12) Patent Application Publication
(10) Pub. No.: US 2022/0378328 A1
(43) Pub. Date: Dec. 1, 2022

(54) INSULATING BULKHEAD COVER

(71) Applicant: Thomas Derrick, Holden, MA (US)

(72) Inventor: Thomas Derrick, Holden, MA (US)

(21) Appl. No.: 17/329,927

(22) Filed: May 25, 2021

(51) Int. Cl. A61B 5/11; G01B 5/02

(52) U.S. Cl. CPC A61B 5/1121; G01B 5/02; A61B 2505/09

(57) ABSTRACT

A device or system including an assessment device with a body with a top surface ruled with a pattern to or instruct students in their parts during a physical position or movement between different positions, such as may be learned in a class or virtual class. The rulings include vertical lines and horizontal lines at various spaces, spacings, and intersections.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of insulation of existing art and more specifically relates to bulkhead insulating solution.

### RELATED ART

Residential cellars are commonplace in some areas. Cellars may be multipurpose, or they may have a single purpose which may determine how it is designed. Most cellars have an entry exit to the outside. Often, in a home where the basement is below ground level, this is provided in the form of a horizontal or sloping structure attached to the exterior of the cellar where the entryway breaks ground. Often, the structure is framed exteriorly to the foundation perimeter. This structure frames and supports a pair of doors that open outwardly from the structure and provide access to the entry stairwell. This structure is known as a bulkhead.

In cold climates the bulkhead, or cellar doors, are an area of major heat loss. Portions of the basement above ground level are areas of concern for heat loss of the house, but the bulkhead doors are generally the biggest concern. One reason why is there are many options for insulating the area, but the options used should not obstruct egress from the basement which limits choices, and the standard options are relatively expensive and/or temporary. Being above the cellar, heat naturally rises to the bulkhead and passes through the thin wooden doors. Bulkheads are also made in many different dimensions further hindering a common dimension standard, making a modular industry solution challenging.

During construction, existing market bulkhead doors can be planned for but in an older home or one that has already been constructed, a homeowner is forced to seek a retrofitted solution. The retrofit is then either homemade or the result of a time-consuming construction project. Suitable retrofits are difficult to find, as binding insulation to the interior of the door interferes with latching and access hardware, and affixing insulation to the exterior presents weatherproofing issues, insulation (such as fiberglass) generally not being waterproof. Accordingly, there is perceived a need for an insulating solution applicable to swinging bulkhead doors, and which is easily customizable to fit any size of bulkhead door. This solution should be weatherproof and easy to install.

Various attempts have been made to solve problems found in bulkhead insulation art. Among these are found in: U.S. Pat. No. 6,941,709 to Denver L. Stanford, Sr., U.S. Pub. No. 2006/0154115 to Thomas Fellinger, U.S. Pat. No. 5,600,921 to William L. Vardaro, U.S. Pat. No. 8,062,985 to Robert P Collier, U.S. Pat. No. 4,675,225 to William P. Cutler, and U.S. Pat. No. 6,551,677 to Larry J. Weinstein. This prior art is representative of insulating devices for bulkhead doors. For example, U.S. Pat. No. 6,941,709 to Stanford teaches a door insulator including a door covering and insulator device for selectively positioning over exterior positioned lower-level doors. The device comprises a first panel and a second panel that each has a size and shape substantially equal to a size and shape of the doors. However, Stanford's device fails to provide suitable weatherproofing and insulation properties for some applications, having only an insulating layer and a waterproofing applied to the exterior of the covering. A superior solution is still sought.

### SUMMARY OF THE INVENTION

In view of the foregoing disadvantages inherent in the known insulation art, the present disclosure provides a novel bulkhead insulating cover. The general purpose of the present disclosure, which will be described subsequently in greater detail, is to provide a bulkhead insulating cover.

A bulkhead insulating cover for insulating and weatherproofing bulkhead doors includes three layers. The first layer, intended to directly contact with an exterior of a bulkhead door, is constructed of a flash-spun high-density polyethylene fiber fabric, and reduces airflow and water ingress, primarily functioning as a vapor barrier. The second layer is a substrate of reflective insulation material composed of an aluminum foil substrate. Air capsules may be integrated within this second layer to provide additional insulating capability. The third layer, to be faced outwardly as the primary weatherproofing layer, is composed of polyester material. The insulating bulkhead cover may be installed by using a hook-and-loop fasteners or attachment means (tradename VELCRO) or magnetic stripping on the inside of the cover.

For purposes of summarizing the invention, certain aspects, advantages, and novel features of the invention have been described herein. It is to be understood that not necessarily all such advantages may be achieved in accordance with any one embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a bulkhead insulating cover, constructed and operative according to the teachings of the present disclosure.

FIG. 1 is a perspective view of a cellar door insulating blanket system showing a blanket assembly lying flat according to an embodiment of the present invention.

FIG. 2 is a cross-sectional view illustrating the cellar door insulating blanket system comprising a multi-layer insulation blanket assembly according to an embodiment of the present invention.

The various embodiments of the present invention will hereinafter be described in conjunction with the appended drawings, wherein like designations denote like elements.

### DETAILED DESCRIPTION

As discussed above, embodiments of the present disclosure relate to an insulator and more particularly to an insulating bulkhead cover as used to improve the heat retention and weatherproofing of a cellar bulkhead.

An insulating bulkhead cover provides a blanket-like device able to be fastened to bulkhead doors to cover and insulate them, reducing the magnitude of heat loss from an interior of a cellar through the bulkhead. The insulating cover may also be referred to as a bulkhead boot or bulkhead blanket. When installed properly, the cover may be capable of reducing heat transference to the outside to a rate of about 97% efficiency.

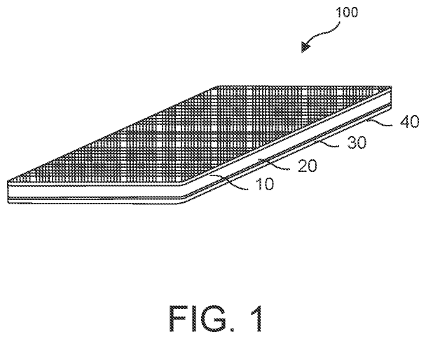

Referring now to the drawings, FIG. 1 shows a cellar door insulating blanket system 100 according to an embodiment of the present invention. Existing cellar door insulating products on the market generally are either of too low of an efficiency, difficult to retrofit to an existing bulkhead door arrangement, or require reconstruction of the bulkhead or bulkhead door framework. In colder climates, energy efficiency is a higher priority than in milder climates. Bulkhead doors, or cellar doors, are one of the largest areas for heat to escape from a dwelling. A particular problem is existing bulkhead doors that already have low energy efficiency. The current invention is a multi-layered blanket that should reduce heat transference to the outside to about 97% efficiency. The bottom layer is constructed of a synthetic weatherproof layer made up of flash-spun high-density polyethylene fibers 40. This bottom layer (or first layer) 40 provides material strength to the bulkhead insulator, provides a vapor barrier, and generally provides waterproofing and a resistance to air drafts. Furthermore, as an airflow barrier, the bottom layer 40 enhances the ability of the insulating layers to work. A second layer 30 is bound to the first layer 40. Second layer 30 may be a compound layer and may include at least one aluminum foil substrate. In a preferred embodiment, second layer 30 comprises a first-aluminum foil layer, a second-aluminum foil layer, and a polyethylene layer sandwiched between the first-aluminum foil layer and the second-aluminum foil layer. Second layer 30 may also include bubble wrap 20, preferably with a large size of bubble. A third layer 10 is bound to second layer 30, such that second layer 30 is sandwiched between first layer 40 and third layer 10. Third layer 10 may include a type of polyester material that is very strong, yet durable and easy to use. This exterior polyester material is to be faced outwardly when the bulkhead insulator is installed, serving as the primary weatherproofing layer, preventing the ingress of water and airflow to the second layer 30, which serves as the primary insulating layer. The assembled insulating blanket would be installed by using a series of fasteners attached to the first layer 40, which may include hook-and-loop fasteners or attachment means (tradename VELCRO) or a magnetic stripping on the inside of the cover for metal bulkhead applications.

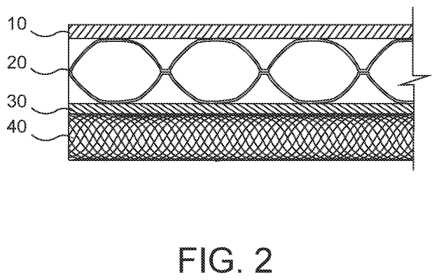

Referring now to FIG. 2 showing a profile diagram illustrating the cellar door insulating blanket 100 (referenced in FIG. 1) according to an embodiment of the present invention. The view in FIG. 2 depicts a cutaway view of the individual layers that make up the cellar door insulating blanket 100. The top layer that may be exposed to the elements is polyester material of third layer 10 that adds strength and durability to the blanket, and provides protection to the inside, less durable materials. Bubble wrap (or bubble pack) 20 may be of an industrial grade that will provide some resiliency to continued use such as handling, rolling, storing, and deploying for over a long period of time. Bubble wrap may be defined as a flexible layer of arrayed, evenly-spaced, air-filled sealed chambers. The third layer 10 may be referred to as a space blanket. The material is a reflective insulation material called an aluminum foil substrate that can reflect a high percentage of low temperature air that manages to penetrate the first two layers. The first layer 40 (the flash-spun high-density polyethylene fibers layer) is the final insulating layer. This layer is more of a synthetic material made up of flash-spun high-density polyethylene fibers that also provides some resistance to continued handling as well as having insulating properties. Cellar door insulating blanket 100 is versatile for use on various types of doors and may be sized for any width or length of door. Layers 10, 20, and 40 may be laminated together, woven together, or bound together by some other mechanism. The exact specifications, materials used, and method of use of the cellar door insulating blanket system may vary upon manufacturing.

It should also be noted that, under appropriate circumstances, considering such issues as design preference, user preferences, marketing preferences, cost, structural requirements, available materials, technological advances, etc., other methods for insulating a bulkhead, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A guidance device comprising:
a body having
a top surface including rulings comprising
three full-length vertical lines (FLVL), left, right, and middle FLVL,
wherein
the middle FLVL divides the left side of the guidance device from the right side,
the left and right FLVL are arranged symmetrically with respect to the middle FLVL,
and
the left FLVL is arranged asymmetrically with respect to the left side of the guidance device,
and
three full-length horizontal lines (FLHL), far, near, and middle FLHL
and
a bottom surface,
wherein the rulings are adapted to provide body position targets to the user.

2. The device of claim 1, wherein the rulings are adapted to provide student performance data to an instructor.

3. The device of claim 1, wherein the middle FLHL divides the far half of the guidance device from the near half.

4. The device of claim 3, wherein
the near and far FLHL are arranged symmetrically with respect to the middle FLHL
and
the near FLHL is arranged asymmetrically with respect to the near half of the guidance device.

5. The device of claim 4 further comprising a first auxiliary FLHL between the near FLHL and the middle FLHL.

6. The device of claim 5, wherein the rulings further comprise first movement rulings symmetrically spaced from the middle FLVL.

7. The device of claim 6, wherein the first movement rulings are spaced 4″ from the middle FLVL and labeled with a total distance between the rulings.

8. The device of claim 7, wherein the rulings further comprise second movement rulings symmetrically spaced from the middle FLVL.

9. The device of claim 8, wherein the second movement rulings are spaced 3″ from the middle FLVL and labeled with a total distance between the rulings.

10. The device of claim 9, wherein the rulings further comprise two FLHL asymmetrically located in the far half of the guidance device.

11. The device of claim 10, wherein the rulings further comprise another FLHL in the far half of the guidance device.

12. The device of claim 11, wherein the rulings further comprise two FLHL asymmetrically located in the near half of the guidance device.

13. The device of claim 12, wherein the rulings further comprise another FLHL in the near half of the guidance device.

14. The device of claim 13, wherein one of the FLHL located in the near half is 12 inches from the bottom edge.

15. The device of claim 14, wherein one of the FLHL located in the near half is 17 inches from the bottom edge.
